(19) United States
(12) Patent Application Publication
(10) Pub. No.: US 2010/0210926 A1
(43) Pub. Date: Aug. 19, 2010

(54) Apparatus and Method for Detecting at Least One Vital Parameter of a Person; Vital Parameter Detection System

(76) Inventors: Fabio Ciancitto, Erlangen (DE); Andreas Tobola, Hemhofen (DE); Christian Hofmann, Nuernberg (DE); Robert Couronne, Erlangen (DE)

Correspondence Address: GLENN PATENT GROUP, 3475 EDISON WAY, SUITE L, MENLO PARK, CA, 94025, US

(21) Appl. No.: 12/703,563

(22) Filed: Feb. 10, 2010

(30) Foreign Application Priority Data
Feb. 12, 2009 (DE) 102009008604.8

(51) Int. Cl. A61B 5/1455

(52) U.S. Cl. 600/310

(57) ABSTRACT

What is described is an apparatus for detecting at least one vital parameter of a person, including: an optoelectronic sensor arrangement for detecting the at least one vital parameter by means of light transmission or light remission, the optoelectronic sensor arrangement including a light source and a light-sensitive element, wherein for detecting the vital parameter by means of light transmission, the light source is arranged in a first side part of a support frame of a pair of spectacles, and the light-sensitive element is arranged in a second side part of the support frame which is opposite the first side part, and wherein for detecting the at least one vital parameter by means of light remission, the light source and the light-sensitive element are arranged in the same side part of the support frame of the pair of spectacles.

### CROSS-REFERENCE TO RELATED APPLICATIONS

This application claims priority from German Patent Application No. 102009008604.8, which was filed on Feb. 12, 2009 and is incorporated herein in its entirety by reference.

The present application relates to a measuring apparatus and to a method of monitoring one or several vital parameters of a person, e.g. detecting an arterial plethysmogram, the heart rate, the heart rate variability, the oxygen content of the arterial blood, and the pulse wave transmission time.

### BACKGROUND OF THE INVENTION

Known methods of detecting vital parameters comprise optical plethysmography and pulse oximetry, for example. Optical plethysmography and pulse oximetry are based on similar measuring methods. They consist in an active sensor apparatus which contains a light source and a photo receiver and is designed such that light passes the tissue layers, and that the remaining light intensity is measured by the photo receiver. When the light passes the tissue layer, it undergoes attenuation which is dependent, among other things, on the wavelength of the light, on the type and concentration of the substances within the irradiated tissue, and on the volume changes in the arterial bloodstream. The photo receiver converts the impinging light to a photocurrent, the amplitude of which is modulated by the volume changes in the arterial vessels, which are caused by myocardial contractions.

Optical pulse oximeters and optical plethysmographs are typically attached to the finger or earlobe of the patient, because in said places, the upper skin layers are densely interspersed with arterial blood vessels, and the attenuating influence of bone or fat tissue is at a minimum. The plethysmographs employed are both those based on the transmission principle and those based on the remission principle. For the remission method, the finger is not entirely irradiated as for the transmission method, but that light portion which is emitted, or, in other words, reflected or remitted, by the tissue following the irradiation with light, is measured. Restriction of the patient's freedom of movement is common to all of the optical plethysmographs and pulse oximeters for being used on a finger, for example attached to the fingertip by means of a finger clip. In addition, such sensors exhibit a very sensitive reaction to low blood circulation and/or vasoconstriction of the peripheral arterioles, which considerably complicates taking and evaluating the plethysmogram and vital parameters that may be derived, such as heart rate, heart rate variability, oxygen content of the arterial blood, and pulse wave transmission time.

In other methods, the measurement is performed in that the source of light is inserted into a nostril of a patient, and a light sensor is inserted into the other nostril, so as to perform the measurement on the basis of the bloodstream within the nasal septum.

### SUMMARY

According to an embodiment, an apparatus for detecting at least one vital parameter of a person may have: an optoelectronic sensor arrangement for detecting the at least one vital parameter by means of light transmission, the optoelectronic sensor arrangement including a light source and a light-sensitive element, the light source being arranged in a first side part of a support frame of a pair of spectacles, and the light-sensitive element being arranged in a second side part of the support frame which is opposite the first side part.

According to another embodiment, an apparatus for detecting at least one vital parameter of a person may have: an optoelectronic sensor arrangement for detecting the at least one vital parameter by means of light remission, the optoelectronic sensor arrangement including a light source and a light-sensitive element, the light source and the light-sensitive element being arranged in the same side part of the support frame of the pair of spectacles.

According to another embodiment, a method of detecting at least one vital parameter of a person by means of an optoelectronic sensor arrangement, wherein the optoelectronic sensor arrangement includes a first number of light sources and a second number of light-sensitive elements, a light source of the first number of light sources being allocated to a light-sensitive element of the second number of light-sensitive elements in each case and forming with same a selectable measurement allocation of a third number of selectable measurement allocations, may have the steps of: performing at least one optoelectronic measurement for the selectable measurement allocations so as to generate at least one measurement result in each case; determining an amplitude-dependent measurement quality for each of the selectable measurement allocations on the basis of the at least one respective measurement result; selecting that measurement allocation of the third number of measurement allocations which has the highest amplitude-dependent measurement quality; and performing at least one optoelectronic measurement by means of the selected measurement allocation so as to detect the at least one vital parameter on the basis thereof

According to another embodiment, a vital parameter detection system may have: an apparatus for detecting at least one vital parameter of a person, which apparatus may have: an optoelectronic sensor arrangement for detecting the at least one vital parameter by means of light transmission, the optoelectronic sensor arrangement including a light source and a light-sensitive element, the light source being arranged in a first side part of a support frame of a pair of spectacles, and the light-sensitive element being arranged in a second side part of the support frame which is opposite the first side part; and a controller configured to perform a method of detecting at least one vital parameter of a person by means of an optoelectronic sensor arrangement, wherein the optoelectronic sensor arrangement includes a first number of light sources and a second number of light-sensitive elements, a light source of the first number of light sources being allocated to a light-sensitive element of the second number of light-sensitive elements in each case and forming with same a selectable measurement allocation of a third number of selectable measurement allocations, which method may have the steps of: performing at least one optoelectronic measurement for the selectable measurement allocations so as to generate at least one measurement result in each case; determining an amplitude-dependent measurement quality for each of the selectable measurement allocations on the basis of the at least one respective measurement result; selecting that measurement allocation of the third number of measurement allocations which has the highest amplitude-dependent measurement quality; and performing at least one optoelectronic measurement by means of the selected measurement allocation so as to detect the at least one vital parameter on the basis thereof.

According to another embodiment, a vital parameter detection system may have: an apparatus for detecting at least one vital parameter of a person, which apparatus may have: an optoelectronic sensor arrangement for detecting the at least one vital parameter by means of light remission, the optoelectronic sensor arrangement including a light source and a light-sensitive element, the light source and the light-sensitive element being arranged in the same side part of the support frame of the pair of spectacles; and a controller configured to perform a method of detecting at least one vital parameter of a person by means of an optoelectronic sensor arrangement, wherein the optoelectronic sensor arrangement includes a first number of light sources and a second number of light-sensitive elements, a light source of the first number of light sources being allocated to a light-sensitive element of the second number of light-sensitive elements in each case and forming with same a selectable measurement allocation of a third number of selectable measurement allocations, which method may have the steps of: performing at least one optoelectronic measurement for the selectable measurement allocations so as to generate at least one measurement result in each case; determining an amplitude-dependent measurement quality for each of the selectable measurement allocations on the basis of the at least one respective measurement result; selecting that measurement allocation of the third number of measurement allocations which has the highest amplitude-dependent measurement quality; and performing at least one optoelectronic measurement by means of the selected measurement allocation so as to detect the at least one vital parameter on the basis thereof.

An embodiment of the present application provides an apparatus for detecting at least one vital parameter of a person, comprising: an optoelectronic sensor arrangement for detecting the at least one vital parameter by means of light transmission or light remission, the optoelectronic sensor arrangement comprising a light source and a light-sensitive element, wherein for detecting the vital parameter by means of light transmission, the light source is arranged in a first side part of a support frame of a pair of spectacles, and the light-sensitive element is arranged in a second side part of a support frame which is opposite the first side part, so that when the glasses are placed on the person's nose, the light of the light source may irradiate the nose for detecting the vital parameter, and for detecting the vital parameter by means of light remission, the light source and the light-sensitive element are arranged in the same side part of the frame, so that when the glasses are placed on the person's nose, the light-sensitive element may receive a portion of the light generated by the light source, said portion being reflected by the nose.

Such an integration of the optoelectronic sensor arrangement into a pair of spectacles enables measuring the vital parameter on the basis of the blood flow of the dorsal nasal artery and/or the angular artery, and therefore is hardly affected by vasoconstriction.

Vasoconstriction appears, for example, in stressful situations, situations of low blood pressure or of hypothermia. The vessels of the legs and arms are constricted so as to achieve improved blood circulation, or a higher blood pressure, in the vital organs, e.g. the brain and the heart. The dorsal nasal artery and the angular artery are direct derivations, or branches, of the internal cervical artery and are therefore less affected by vasoconstriction than the usual points of measurements such as finger, toe or earlobe, for example. Therefore, embodiments enable reliable detection of the vital parameters even in the above-mentioned particular situations.

A further embodiment of the present invention of transmission measurement is characterized in that the support frame of the pair of spectacles is rigid, and the light source and the photosensor have a fixed mutual geometric arrangement defined by the support frame. In this manner, distortion of the measuring results which is due to relative motion of the light source with regard to the light-sensitive element is reduced.

In accordance with a further embodiment of detecting by means of light transmission, the photosensor is arranged, in the one side part of the support frame, such that a direction of a maximum light output of the first photosensor corresponds to the shortest path of the light from the first photosensor to the light element in the oppositely located second side part of the support frame. This enables reducing the energy requirement of the sensor arrangement while keeping the quality of measurement at the same level, or an increase in the quality of measurement while keeping the same energy supply at the same level.

In a further embodiment of the present invention, the optoelectronic sensor arrangement comprises several light sources and light-sensitive elements which are arranged, in a spatially distributed manner, in the side part(s) of the support frame, and are mutually allocated in pairs, and additionally a control means designed to select, from said several pairs of light sources and light-sensitive elements, that pair which provides a higher quality of measurement as compared to the other pairs. Thus, that measurement pair may be selected which is located closest to an artery in the state of the pair of spectacles being worn, and thus, the quality and reliability of the detection of the vital parameters may be improved.

### BRIEF DESCRIPTION OF THE DRAWINGS

Embodiments of the present invention will be detailed subsequently referring to the appended drawings, in which:

FIG. 1 shows a schematic representation of an apparatus for detecting at least one vital parameter of a person, when the apparatus is placed on the person's nose.

FIG. 2 shows an exemplary curve of an optoelectronic measurement signal generated by an embodiment of an apparatus for detecting at least one vital parameter of a person.

FIG. 3 shows a further embodiment of an apparatus for detecting at least one vital parameter of a person.

FIG. 4 shows an embodiment of an apparatus for detecting at least one vital parameter of a person by means of light remission.

FIG. 5 shows a schematic representation of an apparatus for detecting at least one vital parameter, the apparatus being integrated into the frame of the pair of spectacles.

FIG. 6 shows a schematic representation of the essential candidate arteries for measuring vital parameters.

FIG. 7 shows a schematic representation of an embodiment of an apparatus for detecting at least one vital parameter with a multitude of light sources and light-sensitive elements for performing locally selective measurement for determining the at least one vital parameter.

FIG. 8 shows a flow chart of a method of detecting at least one vital parameter of a person.

FIG. 9 shows a schematic representation of a further arrangement of a multitude of light sources and light-sensitive elements.

FIG. 10 shows a schematic representation of an apparatus, integrated into a pair of spectacles, for detecting at least one vital parameter of a person.

FIG. 11 shows a block diagram of an embodiment of control/evaluation electronics.

Identical reference numerals will be used in the present invention for objects and functional units having identical or similar functional properties.

### DETAILED DESCRIPTION OF THE INVENTION

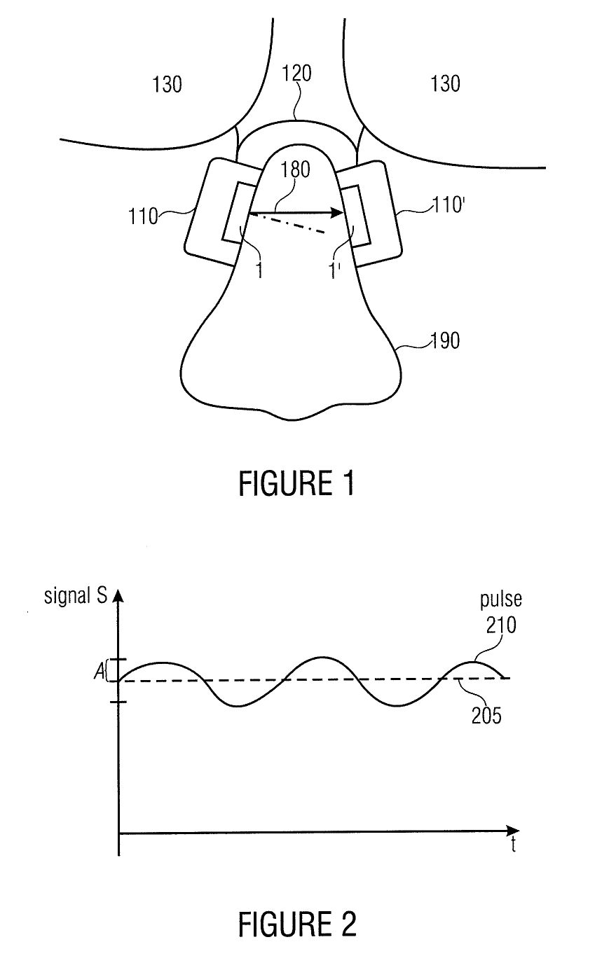

FIG. 1 shows a schematic representation of the apparatus for detecting, wherein the support frame and the optoelectronic sensor arrangement are depicted in a magnified manner so as to be able to better describe the embodiments of the present invention. FIG. 1 shows an embodiment of an apparatus for detecting at least one vital parameter of a person which comprises an optoelectronic sensor arrangement for detecting the at least one vital parameter by means of light transmission, the optoelectronic sensor arrangement comprising a light source 1 and a light sensor 1′, and the light source 1 being arranged in a first side part 110 of the support frame 120 of a pair of spectacles, the light-sensitive 1′ being arranged in a second side part 110′ of the support frame 120, and the second side part 110′ forming a side part of the support frame that is located opposite the first side part 110. FIG. 1 shows the optoelectronic sensor arrangement in a state in which it is placed upon the person's nose. By integrating the light source 1 and the light-sensitive element 1′ into opposite side parts of the support frame 120, the light source and the light-sensitive element are arranged, in a state in which the pair of spectacles is placed on the nose, on opposite sides of the nose or wings of the nose. In accordance with FIG. 1, the light source 1 is arranged on that side of the nose which is on the left-hand side from the viewer's perspective, or, in other words, in the left-hand side part 110 of the support frame, and the light-sensitive element 1′ is arranged on the right-hand side of the nose, or, in other words, in the right-hand side part 110′ of the support frame 120. With regard to the wearer of the pair of spectacles, the side designations “left-hand side” and “right-hand side” are to be used the other way around.

Embodiments of the apparatus further comprise a control means electrically connected to the light source 1 and the light-sensitive element 1′ (not shown) and designed to activate the light source in order to measure a vital parameter, so that said light source generates light of a specific wavelength or wavelength range which radiates through the nose 190 (see arrow 180). Some of the light is absorbed by the tissue layers of the nose 190, so that the light-sensitive element 1′ receives only some of the light, namely the transmitted portion of the light. The degree of absorption or transmission depends on the nature of the tissue and the volume changes in the bloodstream.

FIG. 2 shows an exemplary schematic curve of a measurement signal wherein the static portion 205 (see dashed line) is dependent on the attenuation of the tissue layers, and shows the dynamic, or variable, portion 210, which superimposes said static portion, is dependent on the volume changes in the arterial bloodstream, and is therefore a measure of the pulse.

As may be seen from FIG. 2, an apparatus of FIG. 1 may be used for creating an arterial plethysmogram and for determining the heart, or pulse, rate, the heart rate, or pulse, variability and the pulse amplitude. In addition, in combination with a further measurement at a different location, e.g. the finger, or in combination with an ECG (electrocardiogram), the pulse wave transmission time may be determined.

The vital parameters may be determined, for example, from the amplitude and the amplitude response of the electric signal, e.g. the curve of a photocurrent or a photovoltage, or from a Fourier transformation of said electric signal. Embodiments of the present invention comprise a control or drive circuit which controls the light source 1 with high light intensity, but very short pulses so as to keep the current consumption of the light source low, and comprise, on the receiver side, namely on the side of the light-sensitive element 1′, a peak detector so as to enable reliable digital sampling of the analog plethysmographic signal despite the short light pulses.

Further possibilities of optoelectronic measurement are based on performing a Fourier transformation of the signal measured, rather than on measuring the amplitude.

For detecting the oxygen content (oximetry), one embodiment of the apparatus for detecting comprises two light sources which detect light in light wave ranges which are different from each other. Due to the different coloring of the hemoglobin saturated with oxygen, the two irradiating lights undergo different degrees of absorption, which are measured by the light-sensitive element. An evaluation unit may then determine the oxygen saturation of the blood in the arteries, for example by means of comparing the measurement results with a reference table.

Examples of light sources are light emitting diodes (LEDs), and examples of the light emitting elements are so-called photodiodes. An embodiment that is to measure the oxygen content of the blood may comprise a red diode, which generates visible light in the 660 nanometer range, for example, and an infrared light emitting diode as a further light source which generates a light in the wavelength range of 940 nanometers, for example, which is not visible to humans.

In general terms, the light source is configured to generate light of a first wavelength or wavelength range, and the second light source is configured to generate light of a second wavelength or wavelength range, the first and second wavelengths or wavelength ranges being different from each other.

FIG. 1 further shows the glasses 130 of the pair of spectacles that are mechanically connected to each other via the support frame 120 and rest upon the person's nose 190 by means of the first side part 110 and second part 110′ of the support frame.

The support frame of the pair of spectacles may be configured in a flexible manner in that the distance between the first side part 110 and the second side part 110′ of the support frame changes slightly, for example increases, depending on the width of the nose, when the pair of spectacles is placed on a nose 190, or may be essentially rigid, and therefore the distance between the first side part 110 and the second side part 110′ and, thus, between the light source 1 and the light-sensitive element 1′ essentially does not change.

A rigid implementation of the support frame results in that the light source 1 and the light-sensitive element 1′ have a fixed mutual geometric arrangement defined by the support frame, and therefore results in the risk that the steady component 205 of the signal is changed by a change in the distance between the light source and the light-sensitive element, and that the measurement results are consequently distorted.

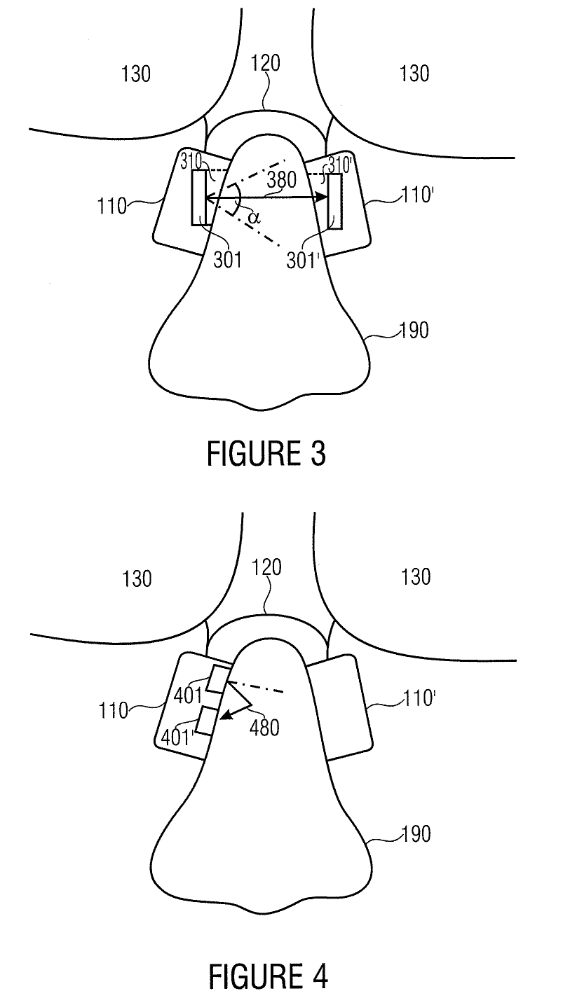

FIG. 3 shows a further embodiment which is similar to that of FIG. 1 but wherein, unlike FIG. 1, the light source 301 is arranged in the first side part 110 of the support frame 120 such that a direction of a maximum light output of the light source corresponds to a shortest path of the light (see reference numeral 380) from the light source 301 to the light-sensitive element 301′. The direction of the maximum light output may also be referred to as the main emission direction.

Embodiments of FIG. 3 comprise light sources, for example, which emit light in an aperture angle α, which light sources typically emit a maximum output in the center of the aperture angle, which may also be referred to as the perpendicular, or perpendicular direction, of the light source (see also arrow with reference numeral 380), and emit less output as the deviation from said perpendicular or said main beam direction increases. This property may be inherent to the light source or be achieved by means of corresponding concentration, e.g. by means of lens structures in the encapsulating material of a light emitting diode. A light source with such focused light emission enables reducing the energy requirement, with measurement results being of similar quality as compared to broader-radiation illumination sources of a larger aperture angle α, or enables, conversely—with the energy requirement being similar—improving the signal amplitude and, thus, also the signal quality.

Accordingly, light-sensitive elements whose degree of efficiency in converting the light received to electric energy is dependent on the direction, are also arranged such that the light generated by the light source 301 is received from this direction (see arrow bearing the reference numeral 380). FIG. 3 further shows a transparent area 310 in the first side part 110 of the support frame, said area 310 being arranged such that the light of the light source 301 may exit in the direction of the nose in an essentially unimpeded manner. Accordingly, the second side part 110′, too, may comprise an area 310 which is transparent at least in the light wave range used, so that the light-sensitive element 301′ may receive the light 380 in an essentially non-attenuated manner.

Corresponding transparent areas 310, 310′ may also be applied in embodiments of FIG. 1.

FIG. 4 shows an embodiment of an apparatus for detecting at least one parameter of a person using an optoelectronic sensor arrangement for detecting the at least one vital parameter by means of light remission, the optoelectronic sensor arrangement comprising a light source 401 and a light-sensitive element 401′, and the light source 401 and the light-sensitive element 401′ being arranged in the same side part of the support frame 120 of a pair of spectacles, or, in other words, are generally arranged, in a state when the pair of spectacles is placed upon the nose, on the same side (here on the first, or left-hand, side from the viewer's perspective) of the nose. The light source 401 and the light-sensitive element 401′ are arranged, for example, within a plane and close to each other. The light source 401 radiates into the tissue of the nose, in which process some of the light is absorbed, some of the light is typically transmitted, and some of the light is generally reflected within the tissue or within the nose. The light-sensitive element receives the reflected remitted portion of the light of the light source 401 (as is symbolically depicted by the case 480). The light source 401 and the light-sensitive element 401′ are mutually arranged in space such that as large as possible a portion of the reflected light of the light source may be received, or that as large as possible a portion of the light of the light source is reflected and may be received by the light-sensitive element 401′.

In alternative embodiments, the light source 401 and the light-sensitive element 401′ may also be arranged on the other side of the nose, for example in the second side part 110′ of the support frame 120, or at another location, e.g. on the bridge of the nose, in that the support frame has an additional resting element there or does not have two discrete side parts 110 and 110′, but a continuous element expanding from the one side of the nose across the bridge of the nose to the other side of the nose.

In this context it shall also be noted that further embodiments of the apparatus for detecting at least one vital parameter may comprise an optoelectronic sensor arrangement for detecting the at least one vital parameter which is configured to determine said at least one vital parameter by means of light transmission and light remission, which may be conducted simultaneously or in an alternating or selectable manner in each case. In other words, the embodiments of FIGS. 1, 3 and 4 may be combined with one another.

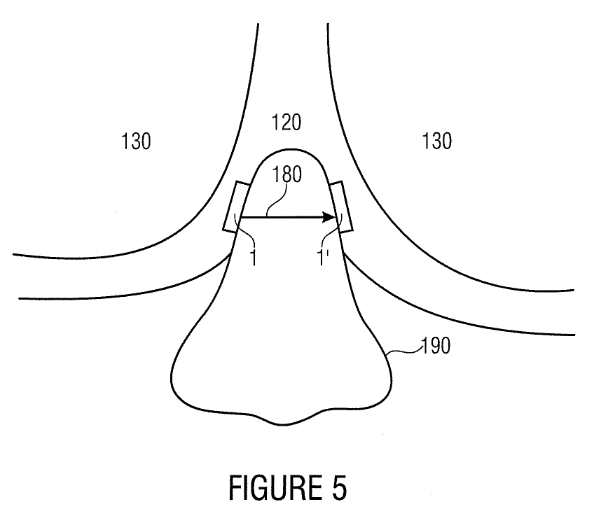

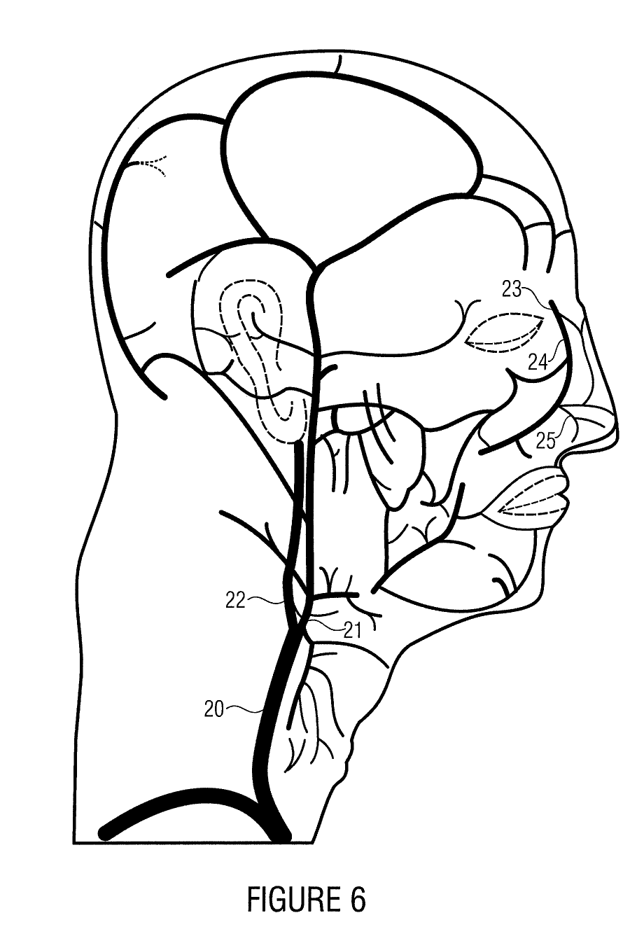

FIG. 5 shows an embodiment of an apparatus for detecting at least one vital parameter which is integrated into a pair of spectacles that does not comprise a separate support frame 120, unlike the embodiments of FIGS. 1, 3 and 4, but wherein the frame of the pair of spectacles itself also is the support frame 120. FIG. 5 depicts the corresponding embodiment of FIG. 1, it being possible for further embodiments to also comprise, e.g., an optoelectronic sensor arrangement of FIG. 3 or 4 or any of those sensor arrangements that will be described in more detail below.

Comparative measurements have shown that the larger the influence of the arteries on the light transmission and light remission, the larger the amplitude A (see FIG. 2) of the modulated pulse signal 210. This influence is at its maximum in the event that the light beam is transmitted through the artery and/or is reflected within it, or, in other words, the smaller the distance between the artery and the light beam trajectory 180, 380 or 480, the larger this influence, and it decreases accordingly as the distance from this light beam trajectory 180, 380 and 480 increases.

FIG. 6 shows the position of the common carotid artery 20, the external carotid artery 21, the internal carotid artery 22, the dorsal nasal artery 23, the angular artery 24, and the lateral nasal artery 25.

Embodiments of the apparatus for detecting at least one vital parameter measure the vital parameters by means of the dorsal nasal artery and/or the angular artery (in the case of light transmission) and a derivation of same (for reflection measurement). Even though the dorsal nasal artery 23 and the angular artery 24 typically have essentially similar curves and similar positions with regard to the nose in all humans, the shape of the nose itself, for example with regard to its height and width, is very different from person to person. Thus, a specific spatial arrangement of the light source 1 and the light sensor 1′ in one side part (reflection measurement) or in both side parts (transmission measurement) of the support frame 120 may be optimally or at least well suited for measuring the vital parameter, whereas due to different nose shapes, the same arrangement of the light source 1 and the light-sensitive element 1′ in the one side part 110 and/or the second side part 110′ may provide less ideal results or even useless results.

Embodiments may therefore comprise, in the side part(s) of the support frame 120, a position and arrangement of the light source 1 and the light-sensitive element 1′ which is specific to the person, i.e. is adapted to the positions and the courses of the arteries, so as to enable optimum measurement in each case.

However, the pair of spectacles may also slide to a different position during wearing and/or may be placed into various positions of the nose when being taken down on or put on again, so that even with such person-specific adaptation of the position and arrangement of the light source and the light-sensitive element degradations of the quality of the measurement results may occur.

Further embodiments of the apparatus for detecting at least one vital parameter of a person comprise a multitude of light sources and a multitude of light-sensitive elements which are mutually allocated in pairs, for example, so that a light source and a light-sensitive element form a measurement pair by means of which a signal S (see FIG. 2) may be measured in order to determine a vital parameter.

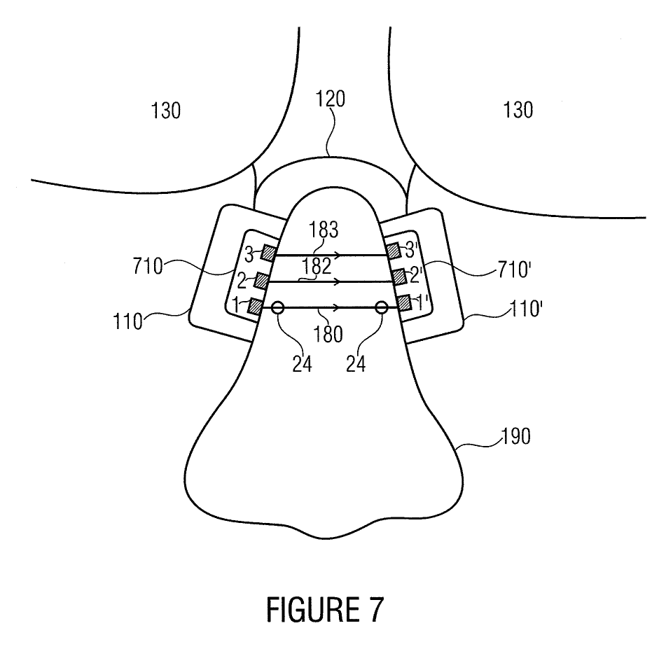

FIG. 7 shows an embodiment of the apparatus comprising a first light source 1, a second light source 2, and a third light source 3 (see hatched areas) in the first side part 110 of the support frame 120 of the pair of spectacles, and a first light-sensitive element 1′ (see hatched areas), a second light-sensitive element 2′, and a third light-sensitive element 3′, which are arranged in the second side part 110′ of the support frame 120 of the pair of spectacles. The first, second and third light sources may be integrated into a component 710 which in turn is integrated into the first side part 110, and the first, second and third light-sensitive elements may be integrated into a component 710′ which in turn may be integrated into the second side part 110′ of the support frame 120, as is shown in FIG. 7. FIG. 7 shows an embodiment wherein the second light source 2 is spatially arranged above the first light source 1 within the component 710 or the first side part 110, and the third light source 3 in turn is spatially arranged above the second light source 2 within the component 710 or in the first side part 110. Accordingly, the second light-sensitive element 2′ is spatially arranged above the first light-sensitive element 1′, and the third light-sensitive element 3′ is spatially arranged above the second light-sensitive element 2′ within the component 710′ or the second side part 110′. In addition, in the embodiment of FIG. 7, the first light source 1 and the first light-sensitive element 1′ are arranged essentially at the same height with regard to their positions relative to the support frame, the second light source 2 is located at the same height as the light-sensitive element 2′, and the third light source 3 is located at the same or essentially at the same height as the third light-sensitive element 3′. In addition, the first light source 1 and the first light-sensitive element 1′ form a first measurement pair, or are allocated to each other for a measurement of detecting the at least one vital parameter, the second light source 2 and the second light-sensitive element 2′ form a second measurement pair, and the third light source 3 and the third light-sensitive element 3′ form a third measurement pair. The apparatus for detecting the at least one vital parameter further comprises a control means (not shown in FIG. 7) which may optionally perform a measurement for detecting the at least one vital parameter by means of the first measurement pair, the second measurement pair or the third measurement pair, the control means being equipped to activate or control the first light source 1 such that the first light source generates one or more light pulses of a specific wavelength or within a specific wavelength range, and to select the first light-sensitive element 1′ to evaluate its signal S or to read out its signal S to a specific evaluation means.

FIG. 7 shows the apparatus for detecting in a state when it is placed onto the nose 190, FIG. 7 depicting the angular artery 24.

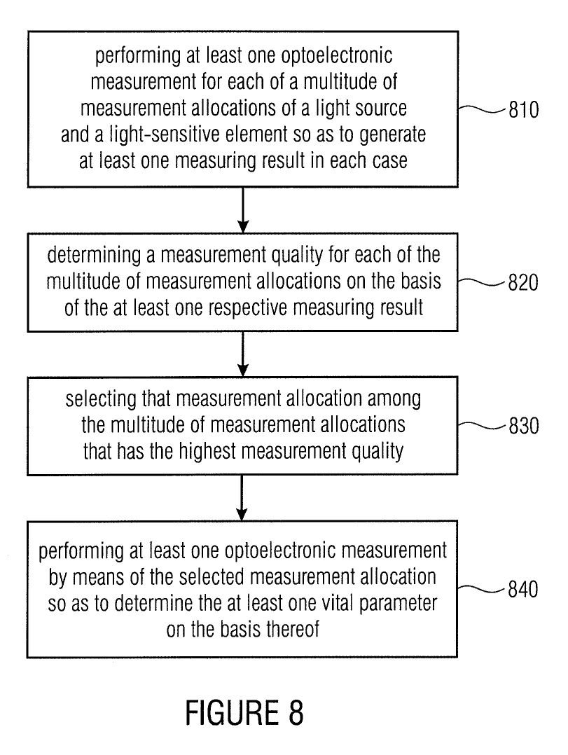

An embodiment of a method of detecting at least one vital parameter of a person by means of an optoelectronic sensor arrangement, (such as in FIG. 7) will be depicted below with reference to FIG. 7 and FIG. 8. The person's vital parameter to be determined is a pulse parameter, e.g. a pulse rate, which is determined on the basis of the sampled amplitude response of the pulse curve 210 as is depicted in FIG. 2. The amplitude of the signal is utilized, at the same time, as a measure of a quality of the optoelectronic measurement signal.

In general terms, a first step of the method comprises selecting a specific measurement pair of the three measurement pairs on the basis of the quality of one or more optoelectronic measurements, which may also be referred to as test measurements, so as to then employ the selected measurement pair for actually determining or detecting the vital parameter in a next step.

FIG. 8 shows a flow chart of an embodiment of a method of determining a vital parameter of a person. This method may be performed, for example, by a control apparatus of the apparatus for detecting the at least one vital parameter or a vital parameter detection system.

Step 810 comprises performing at least one optoelectronic measurement within the same wavelength range or wavelength for each of a plurality of measurement allocations of one light source among a multitude (3 in this case) of light sources and one of a multitude (3 in this case) of light-sensitive elements so as to generate at least one amplitude-dependent measurement result.

Step 820 comprises determining a measurement quality for each of the three or the multitude of measurement allocations on the basis of the respective at least one measurement result.

In step 830, the measurement allocation of the multitude of measurement allocations having the highest measurement quality is selected so as to then perform, in step 840, at least one optoelectronic measurement by means of the selected measurement allocation in order to determine the at least one vital parameter on the basis thereof.

With regard to the apparatus of FIG. 7, for the first measurement allocation, initially the first light source 1 is allocated to the first light-sensitive element 1′, a sufficient number of optoelectronic measurements are performed to determine a minimum and a maximum during the pulse curve 210, and thus, the amplitude of the signal 210. The same is performed for the second measurement allocation consisting of the second light source 2 and the second light-sensitive element 2′, and for the third measurement allocation consisting of the third light source 3 and the third light-sensitive element 3′. For example, a minimum value and a maximum value, respectively, of the signal 210 are determined, and the amplitude A is derived therefrom. At the same time, the amplitude A serves as a measure of quality, meaning that the higher the amplitude, the higher the quality of the signal. This measure of quality may therefore also be referred to as an amplitude-dependent measure of quality. As may be seen in FIG. 7, the light beam 180 for optoelectronic measurement of the first measurement allocation passes through the angular artery and therefore has the highest amplitude. The light beam 182 of the second measurement allocation does indeed not pass through the angular artery, but is still closer to same than the light beam 183 (light path, or path, of the light beam 183) of the third measurement allocation, is therefore influenced more heavily by the pulse of the angular artery 24, and thus has a lower amplitude than the measurement signal of the first measurement allocation, but a higher amplitude than the measurement signal of the third measurement allocation.

As was previously set forth with reference to FIG. 8, step 820 comprises determining the measurement quality, in this case the amplitude, and step 830 comprises selecting the first measurement allocation 1-1′ since it has the highest measurement quality. In step 840, the one or more optoelectronic measurements are then performed so as to determine the person's vital parameter(s). The other measurement allocations 2-2′ and 3-3′ are not employed or are initially not employed.

By putting on and taking off the pair of spectacles, or by moving the pair of spectacles on the bridge of the nose, the position of the selected first measurement allocation 1-1′ relative to the angular artery may change and result in a deterioration of the measurement results.

Further embodiments of the present method are therefore configured to monitor the quality of the measurement signals of the current or active measurement allocation 1-1′, for example to compare it with a threshold value. If the quality of the measurements of the active or currently selected measurement allocation 1-1′ falls below said threshold value, steps 810 to 830 may be performed again, for example, to check whether a different measurement allocation may now provide a higher measurement quality, and to then again perform step 840 for determining the vital parameters.

A further embodiment not only comprises, following step 820, selecting a measurement allocation for the following actual measurements, but also storing the various measurement allocations in an order of priority according to their quality levels, and instead of again performing steps 810 to 830 if the quality threshold value is fallen below by the active measurement allocation, the next or next best measurement allocation, according to the order of priority, is used as the active measurement allocation for the following measurements for determining the vital parameter. In the case of FIG. 7, the measurement allocation 1-1′ is allocated rank 1, the measurement allocation 2-2′ is allocated rank 2, and the third measurement allocation 3-3′ is allocated rank 3. Accordingly, in accordance with the embodiment of the method, the second measurement allocation 2-2′ would be activated next if the quality threshold value was fallen short of by the first measurement allocation. If said second measurement allocation 2-2′ also falls below the quality threshold value the third measurement allocation 3-3′ would be activated, and only if said third measurement allocation 3-3′ also falls below the quality threshold value, steps 810 and 820 will be performed, and the order of priority will be determined.

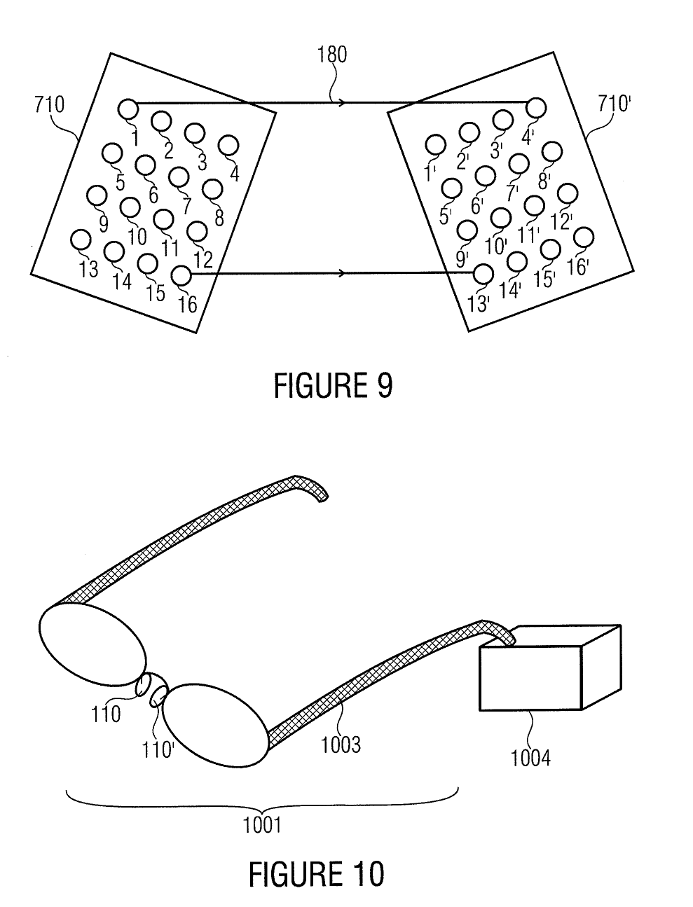

FIG. 9 shows an embodiment of a component 710 having m=16 light sources, and of a second component 710′ also having n=16 light-sensitive elements 1′ to 16′. In accordance with an embodiment of the method, the illumination sources 1 to 16 are allocated their corresponding light-sensitive elements 1′ to 16′ located opposite, respectively, for example the light source 1 and the light-sensitive element 1′ form a first allocation 1 to 1′, the light source 2 and the light-sensitive element 2′ form a second allocation, the light source 3 and the light-sensitive element 3′ form a third allocation, etc. For the transmission method, the light sources and the light-sensitive elements allocated to them are located opposite each other, or are located such that they can detect as large a cross-sectional area of the nose as possible for the measurement. In other words, the light sources and light-sensitive elements that are allocated to each other in each case are located in a mutually symmetrical manner.

Embodiments comprising a first number m of light sources that are spatially arranged in a row, and comprising a corresponding row of light-sensitive elements on the opposite side, enable compensating for any displacement of the pair of spectacles in this very direction. In the embodiment of FIG. 7, a displacement in the pair of spectacles in the vertical direction could be at least partly compensated for.

A planar arrangement of a first number of light sources as is shown, for example, in FIG. 9, enables compensating for the pair of spectacles being displaced in the vertical and horizontal directions relative to the arteries running inside the nose.

A further embodiment will be described on the basis of FIG. 9. Steps 810 and 820 are performed as was previously described with regard to FIG. 8. However, in this embodiment, what is selected is not only the measurement allocation having the highest measurement quality, but at least some, e.g. the 8 best ones, are selected and stored in an order of priority in accordance with their measurement qualities. While performing the optoelectronic measurements, with the selected measurement allocation, on the basis of which the vital parameter is determined, said optoelectronic measurements are simultaneously used for continuously monitoring the quality of the currently selected measurement allocation. If the amplitude-dependent measurement quality of the currently selected measurement allocation falls below a measurement threshold value, the measurement allocation having the next highest measurement quality is selected, in accordance with the order of priority, and the measurements are continued with same so as to detect the vital parameter. The same approach is used when the measurement quality of the next measurement allocation also falls below the measurement threshold value, etc. If the measurement quality of the eighth measurement allocation is also smaller than the measurement threshold value, an optoelectronic measurement will again be performed for all of the 16 measurement allocations, as is described with regard to step 810 and the following.

In embodiments, the light sources and light-sensitive elements may be arranged in arrays, e.g. in a 4×4 array as in FIG. 9 or in any other planar arrangements, they may comprise any number of light sources m with m=1, 2, 3 . . . and any number of light-sensitive elements n=1, 2, 3 . . . The number m of light sources may be equal to the number n of light-sensitive elements, or larger or smaller than same. For example, embodiments may comprise a light source 1 and a multitude of light-sensitive elements, e.g. 1′ to 16′, the multitude of measurement allocations then resulting from different allocations of the one light source to different light-sensitive elements. In an accordingly converse manner, other embodiments may comprise a number n of light sources and only one light-sensitive element. In addition, the same light-sensitive element may be allocated to several light sources, or, in other words, several measurement allocations may comprise the same light-sensitive elements, or vice versa. In addition, a control means may be configured to perform measurements for any potential or for part of any potential allocations so as to variably determine the best measurement allocations. The measurement allocations are not limited to the 1-to-1 allocations as are depicted in FIGS. 7 and 9, but may comprise any other allocations, for example a measurement allocation between the light source 1 and the light-sensitive element 16′ and between the light source 6 and the light-sensitive element 7′. Alternatively, the potential or selectable measurement allocations may be predefined, and the control will only select the best measurement allocations from same.

With regard to their planar arrangement and distribution, the light sources and light-sensitive elements are arranged, in particular, at those positions of the first side part and second side part of the support frame for which it is to be expected, if the pair of spectacles is placed normally on the nose, that at least one of the light paths (e.g. 180, etc) of the different measurement allocations will pass through the artery or will at least have as small a distance as possible to it, so as to achieve as high a quality as possible for measuring the vital parameters.

In further embodiments of the apparatus for detecting, the light sources and light-sensitive elements may be directly integrated, without components 710, 710′, into the first side part 110 or the second side part 110′.

Accordingly, this also applies to embodiments which are based on measurement by means of light remission.

Further embodiments may be configured to also measure the oxygen content of the blood. They will then comprise light sources that may be operated in a different light wave range and that are arranged, for example, next to or between light sources as are depicted in FIGS. 7 and 9. In this case, measurement triples from two light sources having different light wavelengths and a light-sensitive element are defined as measurement allocations. In these embodiments, too, embodiments of the methods described may be applied, e.g. an active measurement triple may be selected for the actual measurements, and other measurement triples may be fallen back on when the quality of the active measurement triple falls below a certain quality threshold value.

In other words, in embodiments of the apparatus for detecting, a multitude of light sources may comprise a first subset of light sources configured to generate light of a first wavelength range, and a second subset of light sources configured to generate light of a second wavelength range different from the first one.

The term “measurement allocation” is generally used for allocations of light sources and light-sensitive elements. The term “measurement pair” designates the specific case where a light source is allocated to a light-sensitive element, e.g. for optical plethysmography, and the term “measurement triple” designates the specific case where a light-sensitive element has two light sources allocated to it which are operated at different wavelengths, e.g. for optical oximetry.

FIG. 10 shows a schematic representation of an embodiment of a measurement apparatus which is integrated into a first side part 110 of the support frame and a second side part 110′ of the support frame of a pair of spectacles 1100. FIG. 10 further shows a control and evaluation unit 1004 comprising the control and evaluation electronics and comprising, e.g., a battery or an accumulator for power supply. The control unit 1004 may be electrically connected, via cables, to the light source(s) and light-sensitive element(s), and/or by conductor traces integrated into the ear pieces 1003, and generally, into the pair of spectacles 1001. The control means 1004 may further comprise a radio module, e.g. Bluetooth-based, for transmitting the vital parameters or statistics on vital parameters, or other information, e.g. control programs or parameters.

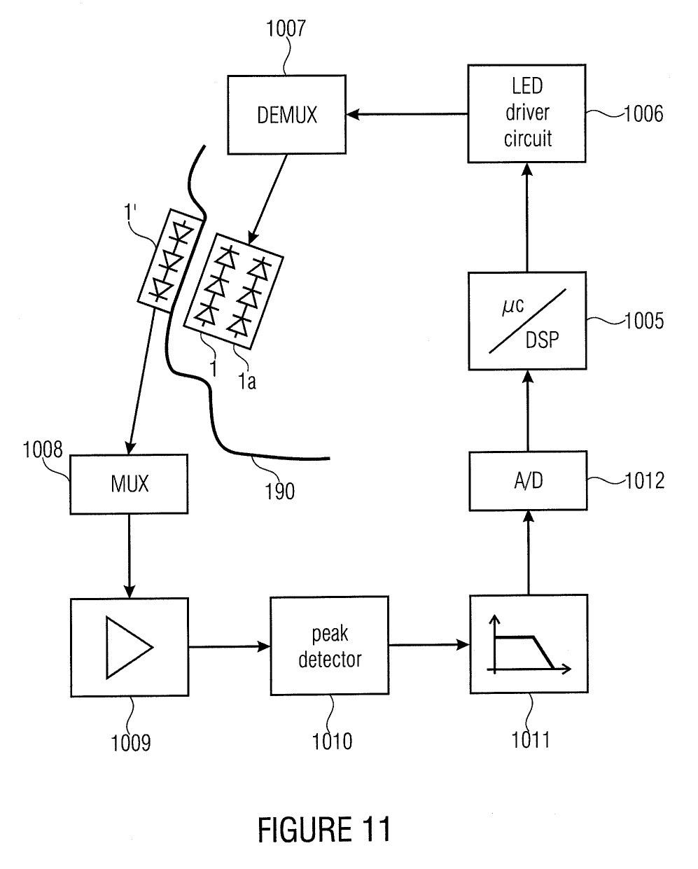

FIG. 11 shows a block diagram of an apparatus for detecting a vital parameter, said apparatus being integrated into a pair of spectacles 1001 (see FIG. 10) into which one or several light sources of different wavelengths, e.g. 1 and 1a, one or several light receivers, e.g. 1′, and a control/evaluation electronics unit 1004 are integrated. By way of example, FIG. 11 shows a light source 1 configured to generate light at a first wavelength, a further light source 1a configured to generate light at a second wavelength which differs from the first one, which are both symbolically depicted by a series connection of light emitting diodes, and a light-sensitive element 1′ on the other side of the nose, symbolically depicted by a series connection of photodiodes. The light sources and the light receiver diodes are attached directly laterally on the bridge of the nose, and the electronics unit 1004 may be attached, for example so as to be spatially separated therefrom and connected by cables, at a less disturbing place upon the body, e.g. the back, the chest, a headband or hat or directly at the earpiece 1003 in an area behind the ear. The control and evaluation electronics unit 1004 comprises, e.g., a microcontroller (pc) and digital signal processor (DSP) 1005, an LED driver circuit 1006, a demultiplexer 1007 in the event of an embodiment wherein various diode allocations may be selected, as previously explained, an analog amplifier 1009, a peak detector 1010, an analog filter 1011, and an analog/digital converter (A/D converter) 1012, and possibly a multiplexer 1008.

In embodiments, a suitable microcontroller, advantageously a digital signal processor 1005, may take over controlling the individual components of the arrangement as well as recording, processing and evaluating the waveforms resulting from the arrangement. The microcontroller, or digital signal processor, controls the LED driver circuit 1006, possibly the demultiplexer 1007, takes over distributing the signals generated to the individual light sources 1, 1a, and selecting, as was described with reference to FIG. 8, measurement allocations in embodiments which enable positional adaptation of the measurement.

Downstream from the analog/digital converter 12, the digitized signal is received and processed by the microcontroller or the digital signal processor 1005. Subsequently, the signal is amplified by means of a circuit 1009, and by means of a peak detector 1010, the pulse is prolonged in terms of time so as to enable improved sampling. Finally, the signal is filtered using a circuit 1011. Thereafter, the plethysmographic signals—this depends on the number of wavelengths—are processed further by the microcontroller or digital signal processor 1005, and vital parameters are calculated from this signal or these signals or in combination with other physiological parameters. Embodiments of the measurement system or of the apparatus for detecting the at least one vital parameter comprise a driver circuit 1006 which may drive the light sources or light emitting diodes at a high light intensity or current, and may operate the light sources 1, 1a at very short pulses, so as to avoid high current consumption of the measurement system, and comprise a peak detector 1010 so as to enable reliable digital sampling of the analog plethysmographic signal.

In view of the above explanations, an object of various embodiments of the present invention is to implement detection of a pulse wave curve, and, derived therefrom, the oxygen saturation of the blood at a location that has so far not been useable, namely the nose, in a non-invasive manner which causes little impairment. Embodiments of the invention enable carrying the measuring means not only in environments of clinics, but also in one's personal surroundings and while being out and about, which is not possible with known finger clip pulse oximeters.

Embodiments of the sensor apparatus are worn like a normal pair of spectacles, so that the restriction on the patient's freedom of movement may be reduced to a minimum. The finger is not inhibited, and the patient is not limited in his/her freedom of movement. The limited or distorted evaluation of important vital parameters, e.g. heart rate, heart rate variability, oxygen content of the arterial blood, and pulse wave transmission time, which is encountered with conventional pulse oximeters and results from low blood circulation or vasoconstriction of the peripheral vessels, is substantially reduced when the nose is used as the place of derivation. The physiological reason for this is that the peripheral vessels, the arterioles, which run within the nasal septum, are direct derivations of the internal cervical artery and are therefore less strongly affected by vasoconstriction than the usual points of measurement, such as on the finger, toe or earlobe.

In other words, embodiments of the present invention enable implementing optical plethysmography and oxygen saturation of the blood derived from several wavelengths, which is integrated into the support frame of a pair of spectacles for metrological application on the bridge of the nose. The apparatus essentially comprises an active optical sensor unit, and the measurement is based on the transmission principle and/or the reflection principle. In yet other words, embodiments of the present invention represent a portable optical plethysmograph and an optical pulse oximeter, which are based on the transmission principle and/or the remission principle, in the form of a pair of spectacles. Accordingly, embodiments may also be referred to as “measurement apparatus for evaluating vital parameters by means of optical transmission or reflection plethysmography on the nasal bone” or “method for detecting vital parameters by means of optical transmission or reflection plethysmography on the nasal bone”.

The field of application of the invention is the field of preventive, monitoring and accompanying medical care for every-day use on a patient's body.

Further embodiments may also be referred to as an apparatus for detecting and evaluating vital parameters by means of optical plethysmography on the bridge of the nose while using transmissive and/or reflective signal acquisition, the apparatus comprising a sensor head which has light sources and light receivers integrated therein and is integrated, on the bridge of the nose, into the support frame of a pair of spectacles, the control and evaluation electronics unit, which is electrically connected to the sensor head and is powered by a battery or an accumulator, being arranged so as to be spatially separated therefrom.

Further embodiments of this apparatus have the shape of a pair of spectacles and evaluate vital parameters by means of optical transmission or reflection plethysmography.

Further developments of these embodiments comprise an optimated driver circuit that may operate light emitting diodes at a high level of light intensity and/or a large amount of current while generating very short light pulses so as to maximize the signal quality and to minimize current consumption.

Even further-reaching developments of these embodiments comprise a peak detector so as to enable reliable digital sampling of the analog plethysmographic signal.

Further embodiments comprise an arrangement of light emitting diodes and photodiodes as transmitters and receivers, which may be switched or selected via multiplexers and demultiplexers, as to be able to vary the position of the measurement area or the point of measurement, so as to thus be able to measure at a location as close to an artery as possible and to therefore be able to select or achieve optimum signal quality.

In addition, embodiments comprise a selection apparatus so as to find and select the optimum position, i.e. a position located as close to an artery as possible, by means of multiplexers and demultiplxers and the arrangement of light emitting diodes and photodiodes.

Depending on the circumstances, the embodiments of the inventive methods may be implemented in hardware or in software. Implementation may be on a digital storage medium, in particular a disk, CD or DVD having electronically readable control signals which cooperate with a programmable computer system such that one of the embodiments of the inventive methods is performed. Generally, the embodiments of the present invention thus also consist in software program products or computer program products or program products having a program code, stored on a machine-readable carrier, for performing one of the embodiments of the inventive methods, when one of the software program products runs on a computer or a processor. In other words, an embodiment of the present invention may thus be implemented as a computer program or a software program or program having a program code for performing an embodiment of an inventive method, when the program runs on a processor.

The processor here may be constituted by a computer, a chip card, a digital signal processor, or any other integrated circuit.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

1. An apparatus for detecting at least one vital parameter of a person, comprising:
an optoelectronic sensor arrangement for detecting the at least one vital parameter by means of light transmission, the optoelectronic sensor arrangement comprising a light source and a light-sensitive element, the light source being arranged in a first side part of a support frame of a pair of spectacles, and the light-sensitive element being arranged in a second side part of the support frame which is opposite the first side part.

2. The apparatus as claimed in claim 1, wherein the support frame of the pair of spectacles is rigid, and the light source and the photosensor comprise a fixed mutual geometric arrangement defined by the support frame.

3. The apparatus as claimed in claim 1, wherein the light source is arranged in the first side part of the support frame such that a direction of a maximum light output of the light source corresponds to a shortest path of the light from the light source to the light-sensitive element.

4. The apparatus as claimed in claim 1, further comprising a controller,
wherein the optoelectronic sensor arrangement comprises a first number of light sources, which may be operated within the same wavelength range, and a second number of light-sensitive elements,
wherein for the transmission measurement, the first number of light sources are arranged in the first side part of the support frame, and the second number of light-sensitive elements are arranged in the second side part of the support frame;
and wherein the controller is configured to select, for detecting the at least one vital parameter, a light source of the first number of light sources and a light-sensitive element, among the second number of light-sensitive elements, which is allocated to the former.

5. The apparatus as claimed in claim 4, wherein the first number of light sources comprise a first subset of light sources configured to generate a light of a first wavelength range, and comprise a second subset of light sources configured to generate a light of a second wavelength range, which is different from the first one.

6. An apparatus for detecting at least one vital parameter of a person, comprising:
an optoelectronic sensor arrangement for detecting the at least one vital parameter by means of light remission, the optoelectronic sensor arrangement comprising a light source and a light-sensitive element, the light source and the light-sensitive element being arranged in the same side part of the support frame of the pair of spectacles.

7. The apparatus as claimed in claim 6, further comprising a controller,
wherein the optoelectronic sensor arrangement comprises a first number of light sources which may be operated within the same wavelength range, and a second number of light-sensitive elements,
wherein for the light remission measurement, the first number of light sources and the second number of light-sensitive elements are arranged in the same side part of the support frame;
and wherein the controller is configured to select, for detecting the at least one vital parameter, a light source of the first number of light sources and a light-sensitive element, among the second number of light-sensitive elements, which is allocated to the former.

8. The apparatus as claimed in claim 7, wherein the first number of light sources comprise a first subset of light sources configured to generate a light of a first wavelength range, and comprise a second subset of light sources configured to generate a light of a second wavelength range, which is different from the first one.

9. A method of detecting at least one vital parameter of a person by means of an optoelectronic sensor arrangement, wherein the optoelectronic sensor arrangement comprises a first number of light sources and a second number of light-sensitive elements, a light source of the first number of light sources being allocated to a light-sensitive element of the second number of light-sensitive elements in each case and forming with same a selectable measurement allocation of a third number of selectable measurement allocations, the method comprising:
performing at least one optoelectronic measurement for the selectable measurement allocations so as to generate at least one measurement result in each case;
determining an amplitude-dependent measurement quality for each of the selectable measurement allocations on the basis of the at least one respective measurement result;
selecting that measurement allocation of the third number of measurement allocations which exhibits the highest amplitude-dependent measurement quality; and
performing at least one optoelectronic measurement by means of the selected measurement allocation so as to detect the at least one vital parameter on the basis thereof.

10. The method as claimed in claim 9, further comprising:
continually determining the amplitude-dependent measurement quality for the selected measurement allocation;
comparing the amplitude-dependent measurement quality with a quality threshold value; and
selecting another selectable measurement allocation of the third number of measurement allocations, or again performing at least one optoelectronic measurement for each of the third number of measurement allocations, and determining the measurement quality for each of the multitude of measurement allocations so as to again select that measurement allocation which exhibits the highest measurement quality, when the amplitude-dependent measurement quality of the currently selected measurement allocation is lower than the quality threshold value.

11. The method as claimed in claim 10, comprising:
following determining an amplitude-dependent measurement quality for each of the measurement allocations, putting at least some of the third number of measurement allocations into an order of priority in accordance with their amplitude-dependent measurement quality;
selecting that measurement allocation of the multitude of measurement allocations which exhibits the highest amplitude-dependent measurement quality;
performing at least one optoelectronic measurement by means of the selected measurement allocation so as to detect the at least one vital parameter on the basis thereof; and
monitoring the quality of, and selecting, that measurement allocation which exhibits the next highest amplitude-dependent measurement quality in accordance with the order of priority when the amplitude-dependent measurement quality of the currently selected measurement allocation is lower than the quality threshold value.

12. A vital parameter detection system comprising:
an apparatus for detecting at least one vital parameter of a person, comprising:
an optoelectronic sensor arrangement for detecting the at least one vital parameter by means of light transmission, the optoelectronic sensor arrangement comprising a light source and a light-sensitive element, the light source being arranged in a first side part of a support frame of a pair of spectacles, and the light-sensitive element being arranged in a second side part of the support frame which is opposite the first side part; and
a controller configured to perform a method of detecting at least one vital parameter of a person by means of an optoelectronic sensor arrangement, wherein the optoelectronic sensor arrangement comprises a first number of light sources and a second number of light-sensitive elements, a light source of the first number of light sources being allocated to a light-sensitive element of the second number of light-sensitive elements in each case and forming with same a selectable measurement allocation of a third number of selectable measurement allocations,
the method comprising:
performing at least one optoelectronic measurement for the selectable measurement allocations so as to generate at least one measurement result in each case;
determining an amplitude-dependent measurement quality for each of the selectable measurement allocations on the basis of the at least one respective measurement result;
selecting that measurement allocation of the third number of measurement allocations which exhibits the highest amplitude-dependent measurement quality; and
performing at least one optoelectronic measurement by means of the selected measurement allocation so as to detect the at least one vital parameter on the basis thereof.

13. A vital parameter detection system comprising:
an apparatus for detecting at least one vital parameter of a person, comprising:
an optoelectronic sensor arrangement for detecting the at least one vital parameter by means of light remission, the optoelectronic sensor arrangement comprising a light source and a light-sensitive element, the light source and the light-sensitive element being arranged in the same side part of the support frame of the pair of spectacles; and
a controller configured to perform a method of detecting at least one vital parameter of a person by means of an optoelectronic sensor arrangement, wherein the optoelectronic sensor arrangement comprises a first number of light sources and a second number of light-sensitive elements, a light source of the first number of light sources being allocated to a light-sensitive element of the second number of light-sensitive elements in each case and forming with same a selectable measurement allocation of a third number of selectable measurement allocations,
the method comprising:
performing at least one optoelectronic measurement for the selectable measurement allocations so as to generate at least one measurement result in each case;
determining an amplitude-dependent measurement quality for each of the selectable measurement allocations on the basis of the at least one respective measurement result;
selecting that measurement allocation of the third number of measurement allocations which exhibits the highest amplitude-dependent measurement quality; and
performing at least one optoelectronic measurement by means of the selected measurement allocation so as to detect the at least one vital parameter on the basis thereof.
